(19) United States
(12) Patent Application Publication
(10) Pub. No.: US 2015/0099952 A1
(43) Pub. Date: Apr. 9, 2015

(54) APPARATUS, SYSTEMS, AND METHODS FOR CARDIOPULMONARY MONITORING

(71) Applicant: Covidien LP, Mansfield, MA (US)

(72) Inventors: David Lain, Easton, MD (US); Gerry Feldman, Atlantic Highlands, NJ (US)

(73) Assignee: COVIDIEN LP, Mansfield, MA (US)

(21) Appl. No.: 14/046,112

(22) Filed: Oct. 4, 2013

(51) Int. Cl. A61B 5/0205; A61B 5/1455

(52) U.S. Cl. CPC A61B 5/0205; A61B 5/14551; A61B 5/024
USPC 600/324; 600/484

(57) ABSTRACT

Systems, methods, and computer executable instructions for monitoring a patient's cardiopulmonary status and/or providing feedback-based control regarding the same. Such methods include, and such systems are configured for, determining a cardiopulmonary status based upon measured data, e.g., from a plurality of sensors, comparing the determined cardiopulmonary status with input data, e.g., input data indicating a target cardiopulmonary status, and providing a result signal based upon the comparison. The result signal may include a display signal for displaying an indication of the result of the comparison, an audio alert signal for audibly provided an indication of the result of the comparison, and/or a control signal for controlling an exercise apparatus in accordance with the result of the comparison.

### TECHNICAL FIELD

The present disclosure relates to monitoring and, more particularly, to apparatus, systems, and methods for monitoring cardiopulmonary function and for providing feedback regarding the same.

### BACKGROUND

Thousands of patients undergo cardiopulmonary rehabilitation every day, whether recovering from surgery, trauma, or injury; combating disease or illness; or attempting to strengthen weakened cardiac and/or pulmonary systems. A typical rehabilitation program may include riding a stationary bicycle, walking on a treadmill, weight lifting, and/or additional physical therapies. While undergoing rehabilitation, caution need be taken to ensure that the cardiac and pulmonary systems are not stressed to the point of further exacerbating the condition being treated. That is, monitoring is performed to ensure that measurable physiological parameters, e.g., heart rate, oxygen saturation, etc., are maintained within acceptable limits for that patient. Typically, such monitoring is performed by trained medical personnel, e.g., doctors, nurses, physical therapists, etc., due to the fact that these trained personnel are generally able to ascertain a condition of the patient based upon the measurable physiological parameters provided.

In addition to those individuals following specific rehabilitation programs, thousands of patients live with weakened cardiac and/or pulmonary systems. For such patients, exercise and even daily activities may detrimentally stress the cardiac and pulmonary systems. Unless admitted to a medical facility or provided with home care, the measurable physiological parameters of these patients are not typically monitored or assessed by medical personnel.

### SUMMARY

The present disclosure relates to apparatus, systems, and methods for monitoring a patient's cardiopulmonary status and/or providing feedback-based control regarding the same. In particular, methods and systems for performing the same provided in accordance with the present disclosure include determining a cardiopulmonary status based upon measured data, e.g., from a plurality of sensors, comparing the determined cardiopulmonary status with input data, e.g., input data indicating a target cardiopulmonary status, and providing a result signal based upon the comparison. The result signal may include a display signal for displaying an indication of the result of the comparison, an audio alert signal for audibly provided an indication of the result of the comparison, and/or a control signal for controlling an exercise apparatus in accordance with the result of the comparison.

The present disclosure is advantageous in that it enables a patient to participate in activities, exercise, follow a rehabilitation program, etc. while being monitored and provided with feedback regarding the patient's cardiopulmonary status. The present disclosure is further advantageous in that the feedback provided to the patient is easy-to-understand and allows the patient to readily ascertain his or her cardiopulmonary status relative to a target status, or target zone. In addition, the present disclosure is advantageous for use with exercise apparatus or other equipment wherein automatic control may be affected and/or operation of the apparatus or equipment limited/inhibited based upon the feedback regarding the patient's cardiopulmonary status.

Certain embodiments of the present disclosure may include some, all, or none of the above advantages and/or one or more other advantages readily apparent to those skilled in the art from the drawings, descriptions, and claims included herein. Moreover, while specific advantages have been enumerated above, the various embodiments of the present disclosure may include all, some, or none of the enumerated advantages and/or other advantages not specifically enumerated above.

### BRIEF DESCRIPTION OF THE DRAWINGS

The present disclosure and its various aspects and features are described hereinbelow with reference to the accompanying drawings, wherein:

FIG. 1 is a schematic illustration of a cardiopulmonary monitoring system provided in accordance with the present disclosure;

FIG. 2 is a flow diagram illustrating a method of cardiopulmonary monitoring provided in accordance with the present disclosure and configured for implementation, for example, using the system of FIG. 1;

FIG. 3A is a side view of the cardiopulmonary monitoring system of FIG. 1 in use in conjunction with an exercise apparatus;

FIG. 3B is a front view of a display board of the exercise apparatus of FIG. 3A.

FIG. 4A is a side view of the cardiopulmonary monitoring system of FIG. 1 in use in conjunction with a portable monitoring apparatus;

FIG. 4B is a top view of a display screen of the portable monitoring apparatus of FIG. 4A;

FIG. 4C is a front view of a smartphone configured for use with the cardiopulmonary monitoring system of FIG. 1;

FIGS. 5A-5C illustrate various display screens provided in accordance with the present disclosure and configured for use with the cardiopulmonary monitoring system of FIG. 1, as presented to a user;

FIG. 5C illustrates another display screen provided in accordance with the present disclosure, as presented to a user;

FIG. 6 is a schematic illustration of another cardiopulmonary monitoring system provided in accordance with the present disclosure; and

FIG. 7 is a flow diagram illustrating a method of cardiopulmonary monitoring provided in accordance with the present disclosure and configured for implementation, for example, using the system of FIG. 6.

### DETAILED DESCRIPTION

Provided in accordance with the present disclosure are apparatus, systems, and methods for monitoring a patient's cardiopulmonary function and for providing feedback and/or feedback-based control based upon the patient's cardiopulmonary status. The feedback is provided in a simplified and readily understandable format, for example, using the Integrated Pulmonary Index (IPI), detailed in U.S. Patent Application Pub. No. 2012/0145152, the entire contents of which are hereby incorporated by reference herein. With respect to feedback-based control, a simplified output, such as that provided via the IPI, is also beneficial in that it reduces the number of control parameters, thus facilitating use with a wide range of controllable or programmable exercise apparatus and other equipment and the customization of control profiles to suit a particular user or group of users. Although detailed herein with respect to the IPI, it is envisioned that the present disclosure be similarly utilized in conjunction with any other suitable system for generating feedback data based upon various inputs.

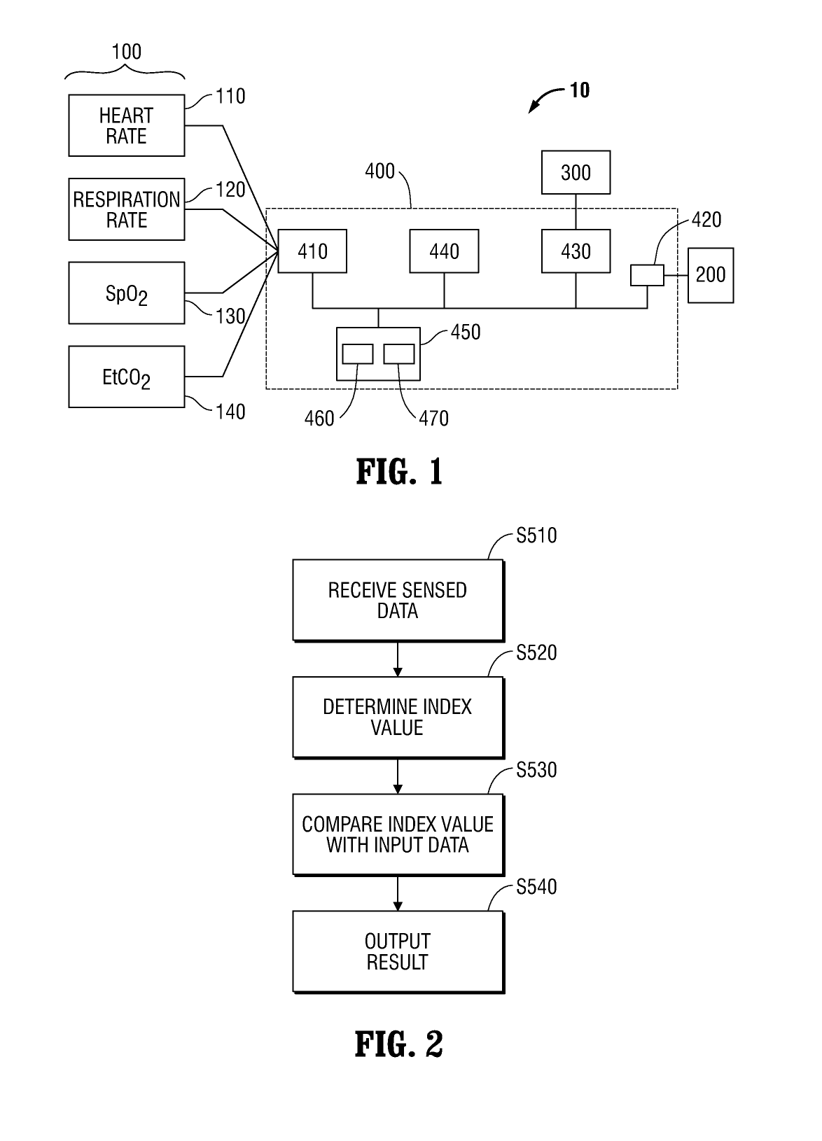

Referring to FIG. 1, an exemplary system for monitoring and providing feedback regarding a patient's cardiopulmonary status provided in accordance with the present disclosure is shown generally identified by reference numeral 10. System 10 includes a plurality of sensors 100, one or more input devices 200, one or more output devices 300, and a processing module 400. For the purposes herein, exemplary system 10 is generally described, although the aspects and features of the present disclosure may be implemented, incorporated, or utilized with any other devices, systems, and combinations thereof.

Sensors 100 may include, for example, a heart rate monitor 110 configured to measure a patient's heart rate, i.e., heart beats per minute, a respiratory rate monitor 120 configured to measure a patient's respiratory rate, i.e., breaths per minute, a pulse oximeter 130 configured to measure a patient's percent oxygen saturation of hemoglobin in arterial blood (SpO₂), and a capnography sensor 140 configured to measure a patient's end tidal carbon dioxide pressure (EtCO₂). Additional or alternative sensors 100 are also contemplated. As detailed below, sensors 100 are configured to communicate with processing module 400 to provide measured data to processing module 400, e.g., heart rate data, respiratory rate data, SpO₂ data, and EtCO₂ data.

The heart rate monitor 110 may be a chest-mounted heart rate monitor secured about the patients chest via a strap, a wrist-worn heart rate monitor, a combination chest-mounted and wrist-worn heart rate monitor, e.g., including a chest-mounted monitoring portion and a wrist-worn display portion, or any other suitable heart rate monitor. The heart rate monitor 110 may be configured to obtain heart rate measurements continuously, at specific intervals (constant or dynamic), and/or upon manual request. The heart rate monitor 110 is configured to communicate with processing module 400 wirelessly, e.g., via Bluetooth® or other suitable wireless communication standard, and/or via a wired connection to provide the measured heart rate data to processing module 400. The heart rate monitor 110 may further include a display for displaying the heart rate data to the patient.

The respiratory rate monitor 120 may be combined or integrated with the heart rate monitor 110. Alternatively, the respiratory rate monitor 120 may be combined or integrated with the capnography sensor 140, or may be a separate, stand-alone component. The respiratory rate monitor 120 may be configured to obtain respiratory rate measurements continuously, at specific intervals (constant or dynamic), and/or upon manual request. The respiratory rate monitor 120 is configured to communicate with processing module 400 wirelessly, e.g., via Bluetooth® or other suitable wireless communication standard, and/or via a wired connection to provide the measured respiratory rate data to processing module 400. The respiratory rate monitor 120 may further include a display for displaying the respiratory rate data to the patient.

The pulse oximeter 130 may be a fingertip-mounted pulse oximeter, or any other suitable monitor configured to measure SpO₂. The pulse oximeter 130 may be configured to obtain SpO₂ measurements continuously, at specific intervals (constant or dynamic), and/or upon manual request. The pulse oximeter 130 may be configured to communicate with processing module 400 wirelessly, e.g., via Bluetooth® or other suitable wireless communication standard, and/or via a wired connection to provide the measured SpO₂ data to processing module 400. The pulse oximeter 130 may further include a display for displaying the SpO₂ data to the patient.

The capnography sensor 140 may be a capnography nasal cannula configured to measure a patient's EtCO₂, such as the wireless capnography sensor detailed in U.S. Patent Application Pub. No. 2011/0066061, the entire contents of which are hereby incorporated by reference herein, or may be any other suitable capnography sensor. The capnography sensor 140 may be configured to obtain EtCO₂ measurements continuously, at specific intervals (constant or dynamic), and/or upon manual request. The capnography sensor 140 is configured to communicate with processing module 400 wirelessly, e.g., via Bluetooth® or other suitable wireless communication standard, and/or via a wired connection to provide the measured EtCO₂ data to processing module 400.

The one or more input devices 200 may include a user-interface of the processing module 400, and/or one or more tablet PCs, smartphones, laptop computers, etc. coupled to the processing module 400, e.g., via a wireless or wired connection. The one or more input devices 200 allow for patient data and/or control data to be provided to processing module 400, e.g., via manual input or automatically pulled from another system such as a patient's Electronic Medical Record (EMR), an Admission, Discharge, and Transfer (ADT) electronic file, lab data, etc.

The one or more output devices 300 may include a user-interface of the processing module 400, and/or one or more tablet PCs, smartphones, laptop computers, etc. coupled to the processing module 400, e.g., via a wireless or wired connection. The input and output devices 200, 300, respectively, may be incorporated into the same device, or maybe separate devices. The one or more output devices 300 are configured to display data output via processing module 400, provide alerts, notifications, reports, etc. output via processing module 400, and/or to effect feedback-based control of, for example, an exercise apparatus, based upon control signals provided via processing module 400.

The processing module 400 may be formed as a stand-alone physical unit, a plurality of interconnected hardware and/or software components (remote and/or local), or partially or fully integrated into a device or system, e.g., an exercise apparatus or portable device. The processing module 400 includes a sensor input 410 configured to receive the measured data, e.g., heart rate data, respiratory rate data, SpO₂ data, and EtCO₂ data, from the sensors 100; a device input 420 configured to receive patient data and/or control data from the one or more input devices 200; an output 430 configured to provide display data, alerts, notifications, reports, etc, and/or control signals to the one or more output devices 300; a storage device 440 configured to store information received via inputs 410, 420, e.g., a magnetic disk, flash memory, optical disk, or other suitable data storage device; and a controller 450. Controller 450 includes a processor 460 and a memory 470. Processor 460 may include any suitable component(s), e.g., a central processing unit (CPU), operable to execute instructions stored in memory 470 to process information, e.g., stored in storage device 440 and/or received via inputs 410, 420, for output to the one or more output devices 300 via output 430. The memory 470 may be any computer memory, e.g., RAM or ROM, mass storage media, removable storage media, combinations thereof, or any other suitable computer-readable storage medium, storing instructions for causing processor 460 to execute particular functions.

With additional reference to FIG. 2, controller 450 is configured to receive the sensed or measured heart rate data, respiratory rate data, SpO₂ data, and EtCO₂ data from sensor input 410 and/or storage device 440 (step S510), and determine an index value, e.g., an IPI value (see the '152 Application Publication, previously incorporated by reference herein), based upon the sensed data (step S520). Patient data input via device input 420 may also be utilized in calculating the index value. The index value may be provided on a scale of 1-10 (10 being optimal) and provides an overall indication of the patient's cardiopulmonary status, by taking into account the above-noted measured data. Although the present disclosure is exemplified using an index scale of 1-10, it is envisioned that any other suitable index scale may be provided.

Controller 450 is further configured to compare the index value with the input data provided via device input 420 and/or storage device 440 (step S530). The input data may include, for example, the patient's last recorded index value, a target index value for the patient, a minimum acceptable index value for the patient, and/or an index value range for the patient. Alternatively, the input data may include medical information about the patient and/or physiological data such as the patient's condition, height, weight, age, etc. Based upon the comparison of the input data and the index value, controller 450 outputs one or more result signals to display data, provide alerts, notifications, reports, etc, and/or output one or more control signals to the one or more output devices 300 (step S540). Thus, as detailed below, the patient, his or her caregiver, a medical professional, etc. may be readily apprised as to the status of the patient's cardiac and pulmonary systems relative to the normal value, target value, or target range provided. Additionally or alternatively, as will also be detailed below, where an exercise apparatus or other equipment utilized by the patient is one of the output devices 300, such apparatus or equipment may be controlled in accordance with control signals provided by controller 450 and based upon the comparison of the input data and the index value. The controller 450 may be configured to perform the above continuously, at pre-determined intervals, and/or upon manual request.

In addition to providing a status of the patient's cardiac and pulmonary systems relative to the normal value, target value, target range, etc., input data provided via device input 420 and/or storage device 440 may also be utilized for determining a level of monitoring required for a patient based upon a baseline value or zone, e.g., normal value, target value, target range, etc., determined from the data or input by a medical professional prior to beginning exercise. The baseline index or zone may be color-coded or otherwise configured to readily indicate a level of monitoring required for the patient based on the severity of the patient's condition. For example, a “red” zone, or relatively low index value may indicate that the patient is to be closely monitored by a medical professional at all times during exercise; a “yellow” zone, or an intermediate index value may indicate that the patient is to be often monitored via a medical professional in close proximity to the exercising patient; and a “green” zone or relatively high index value may indicate that, while support need be provided, the patient need not be closely monitored at all times during exercise. Controller 450 may be configured to output this zone or index information for display, providing alerts, notifications, reports, etc., to alert medical professionals as to the required level of monitoring needed for each particular patient. During use, as the zone or index information changes, controller 450 may output the updated zone or index information for display, providing alerts, notifications, reports, etc., to alert medical professionals as to a change in the required level of monitoring needed.

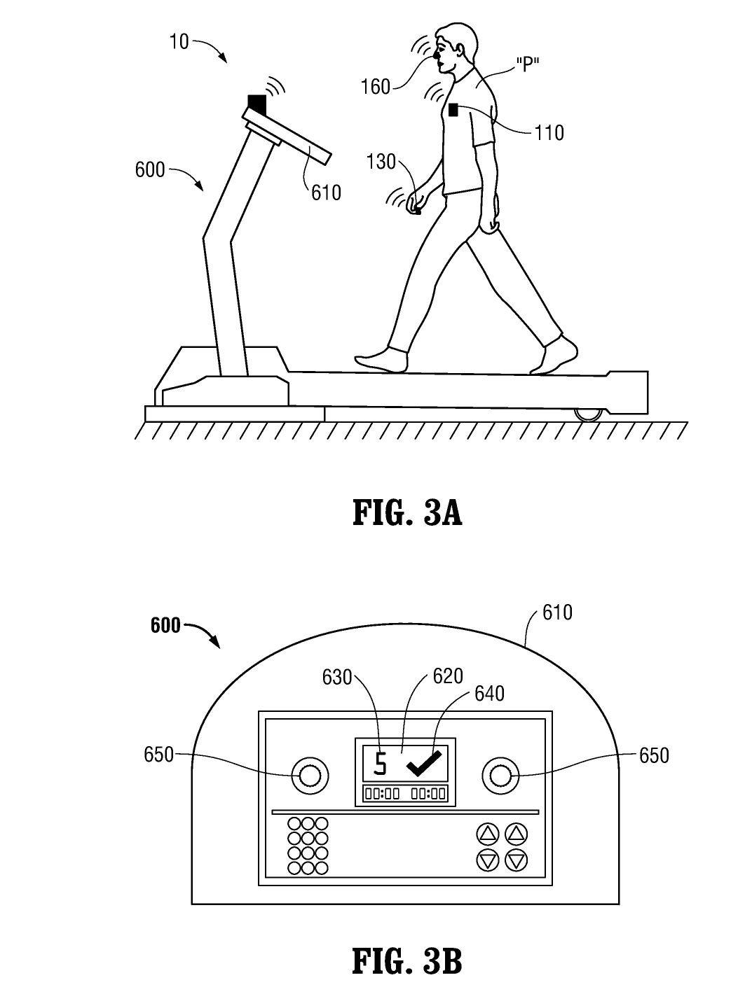

Referring to FIGS. 3A-3B, in embodiments, the processing module 400 (FIG. 1) is integrated, dockable, syncable, etc., with an exercise apparatus such as a treadmill 600, although other exercise apparatus such as, for example, stationary bicycles, rowing machines, elliptical machines, etc. may similarly be used. System 10 may be utilized in conjunction with treadmill 600 during patient rehabilitation, e.g., during the six (6) minute walk test, or for an exercising patient required to monitor his or her cardiac and pulmonary systems. More specifically, with respect to patients having compromised or weakened cardiac and pulmonary systems, it is important to monitor the patient's cardiopulmonary status during exercise to ensure that the patient is exercising enough to achieve benefit, but also to ensure that the patient exercises only in an aerobic zone, as the stress of anaerobic exercise may be too great for the patient to handle and/or may detrimentally harm the patient's already compromised or weakened cardiac and pulmonary systems. Monitoring the cardiopulmonary status during exercise also allows medical personnel to assess a patient's progression through recovery and/or rehabilitation.

In use, the patient “P” is fitted with heart rate monitor 110, nasal cannula sensor 160, which incorporates both the respiratory rate monitor 120 and capnography sensor 140 (FIG. 1), and pulse oximeter 130. As mentioned above, processing module 400 is integrated, dockable, syncable, etc., with treadmill 600. Heart rate monitor 110, nasal cannula sensor 160, and pulse oximeter 130 communicate with processing module 400, e.g., via wireless (such as Bluetooth®) or wired connection, to provide the measured heart rate data, respiratory rate data, SpO₂ data, and EtCO₂ data of the patient “P.”

When system 10 is used in conjunction with treadmill 600, the one or more output devices 300 (FIG. 1) of system 10 may include the display board 610 of treadmill 600. The display board 610 may also function as one of the input devices 200 or output devices 300 (FIG. 1), allowing the user to provided user data and/or control data to processing module 400. Alternatively, user data and/or control data may be provided to processing module 400, as mentioned above, via a tablet PC, smartphone, laptop computer, other suitable device, or may be automatically pulled from the patient's Electronic Medical Record (EMR), an Admission, Discharge, and Transfer (ADT) electronic file, lab data, etc.

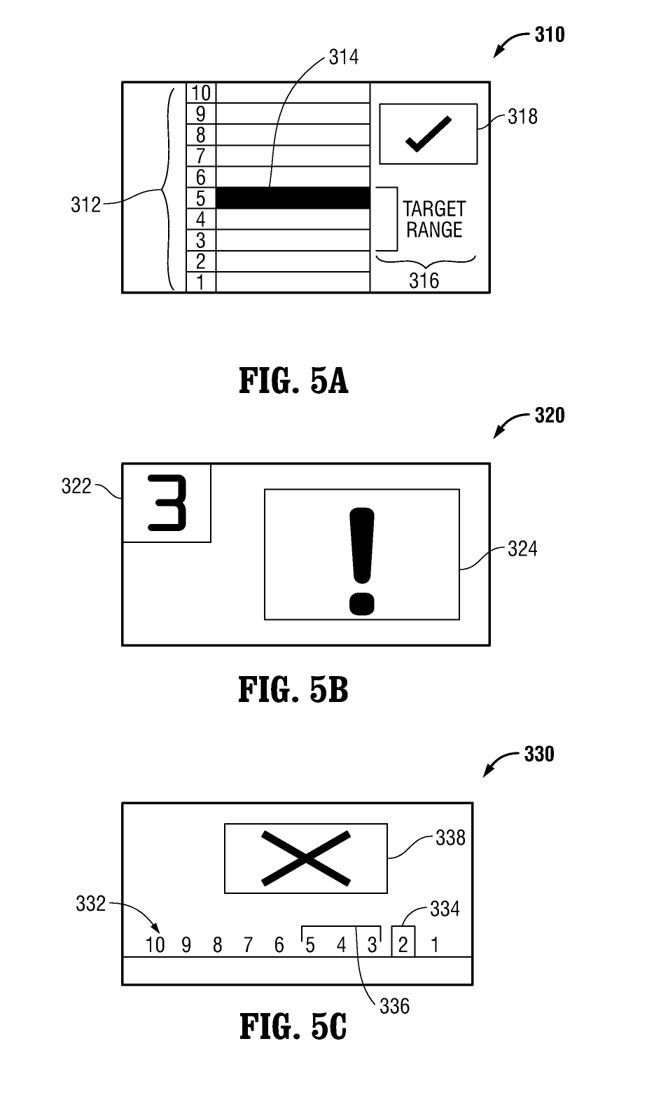

When the patient “P” is exercising on treadmill 600, the processing module 400 (FIG. 1) generates the current index value indicating the status of the cardiac and pulmonary systems of the patient “P,” e.g., continuously, periodically, and/or upon manual request; compares the index value to the input data; and outputs the result to display board 610. FIG. 3B illustrates an exemplary output provided on display board 610. That is, display screen 620 of display board 610 provides the current index value 630 output from the processing module 400 (FIG. 1), e.g., “5,” along with an indicator or symbol 640, e.g., a check mark, indicating the relation between the current index value 630 relative to the normal, target value, or target range provided by the input data (or calculated therefrom). Thus, in the example of FIG. 3B, an index value 630 of “5” is displayed along with check symbol, indicating that the index value 630 is at the normal value, target value, or within the target range. In the exemplary embodiment of FIG. 3B, for example, indicator 640 may provide a green check signal indicating that the index value 630 is acceptable (as shown); indicator 640 may provide a yellow exclamation point where the index value 630 is bordering on or trending away from the normal value, target value, or target range (see FIG. 5B); and indicator 640 may provide a red “X” where the index value 630 is outside of the acceptable value or range (see FIG. 5C). As can be appreciated, various different displays, symbols, indicia, color-coding, etc. may be provided, alone or in combination, to indicate the index value 630 and/or relative status of the patient “P.” Audio alerts, notifications, etc. may additionally or alternatively be provided to the user via speakers 650 of display board 610 of treadmill 600.

Displaying an index value 630 along with an indicator 640 provides a simplified, easy-to-understand indication to the patient “P” regarding his or her cardiopulmonary status. Thus, the patient “P” may readily determine whether further exercise is permitted, or whether the patient “P” should reduce her or her exertion or stop exercising entirely. Various different display features and/or audio effects, such as those noted above, may also be provided to alert the patient “P” as to whether exercise intensity should be decreased or stopped entirely. In some embodiments, the determined index value (step S520 in FIG. 2) is output and displayed, without comparing the value with input data (step S530 in FIG. 2) or displaying the indicator 640. Such embodiments provide for similar benefits as detailed above, without the need to provide specific user data. Systems in such embodiments may thus be generic to all patients and need not be configured to receive user data. Some embodiments may allow the user to select, as an option, whether comparing the value with input data (step S530 in FIG. 2) or displaying the indicator 640 is performed; or may do so only where user data has been provided. Further, as mentioned above, with respect to use in a supervised setting, zone or index information may be displayed, provided as an alert, notification, report, etc., to alert medical professionals as to the required level of monitoring (or changes thereof) for the particular patient.

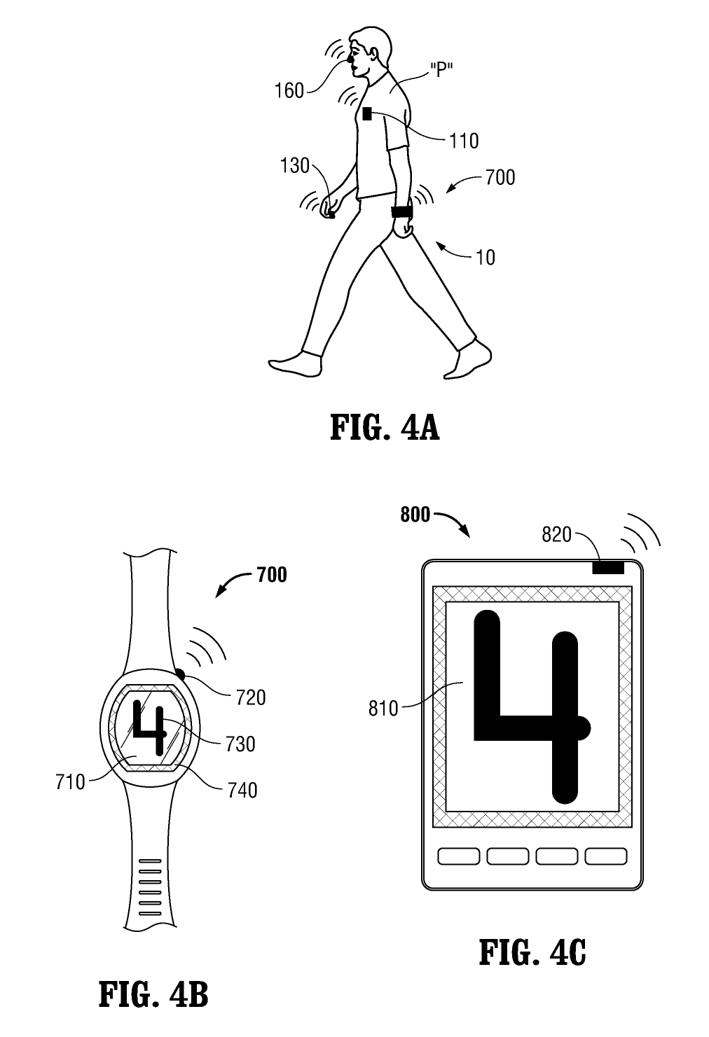

Turning now to FIGS. 4A-4B, in embodiments, the processing module 400 (FIG. 1) is integrated into a portable monitoring device 700, e.g., a wrist-worn device (or monitoring-capable wrist watch). System 10 may be utilized by a patient “P” in conjunction with portable monitoring device 700 during exercise or daily activities. Monitoring the status of the cardiac and pulmonary systems during exercise or daily activities allows the patient “P” to monitor his or her cardiopulmonary status and, if necessary, adjust the level of exercise or activity accordingly.

In use, similarly as detailed above with respect to the previous embodiment, patient “P” is fitted with heart rate monitor 110, nasal cannula sensor 160, which incorporates both the respiratory rate monitor 120 and capnography sensor 140 (FIG. 1), and pulse oximeter 130. The patient “P” further wears the portable monitoring device 700, which may be connected to the heart rate monitor 110, nasal cannula sensor 160, and pulse oximeter 130 via wireless, e.g., Bluetooth®, or wired connection. Portable monitoring device 700 includes a display screen 710 and/or speaker 720 that is utilized as one of the output devices 300 (FIG. 1) of system 10. Similarly as above, when the patient “P” is exercising or performing an activity, processing module 400 (FIG. 1) generates the current index value indicating the status of the cardiac and pulmonary systems of the patient “P;” compares the index value to the input data; and outputs the result to display screen 710 and/or speaker 720.

With particular reference to FIG. 4B, display screen 710 of portable monitoring device 700 provides the current index value 730 output from the processing module 400 (FIG. 1), e.g., “4,” along with an indicator pattern 740 indicating the relation between the current index value 730 relative to the normal, target value, or target range provided. Indicator pattern 740 may include color-coding, symbols, recognizable patterns, flashing, blinking, etc. to indicate whether the index value 730 is within acceptable limits, is bordering on acceptable limits, or is outside acceptable limits. Audio alerts, notifications, etc. regarding the same may additionally or alternatively be provided to the user via speakers 720 of portable monitoring device 700.

Referring to FIG. 4C, as an alternative to portable monitoring device 700 (FIG. 4B), system 10 (FIG. 4A) may be utilized during exercise or daily activities in conjunction with an application (functioning as or integrating processing module 400 (FIG. 1)) stored on or accessible using a smartphone 800 or other suitable device. In such embodiments, smartphone 800 receives the measured data from heart rate monitor 110, nasal cannula sensor 160, and pulse oximeter 140 (FIG. 4A); generates the current index value indicating the cardiopulmonary status; compares the index value to the input data; and outputs the result to display screen 810 and/or speaker 820 of smartphone 800, similarly as detailed above with respect to portable monitoring device 700 (FIG. 4B). Additional features such as those detailed above with respect to treadmill 600 (FIG. 3A) may also be provided for use in conjunction with patient monitoring device 700 (FIG. 4B) and/or smartphone 800.

FIGS. 5A-5C illustrate various different display screens 310, 320, 330 of output devices 300 configured for use with system 10 (FIG. 1). In particular, display screen 310, shown in FIG. 5A, provides an index scale 312 including a highlighted indicator bar 314 indicating the current index value. Display screen 310 further includes a target indicator 316 indicating the target range on the index scale 312, thus allowing a patient to readily ascertain the relative status of his or her cardiac and pulmonary systems. A status indicator 318, similar to indicator 640 (FIG. 3B), may additionally be provided.

Display screen 320, shown in FIG. 5B, is similar to display screen 620 (FIG. 3B), displaying the current index value 322 output from the processing module 400 (FIG. 1), along with a status indicator 324 indicating the relation between the current index value 322 relative to the normal, target value, or target range provided.

As shown in FIG. 5C, display screen 330, similar to display screen 310 (FIG. 5A), provides an index scale 332 including a highlighted selector 334 indicating the current index value. Display screen 330 further includes a target indicator 336 indicating the target range on the index scale 332, and a status indicator 338 similar to indicator 640 (FIG. 3B). Although exemplary display screens 310-330 (FIGS. 5A-5C) are provided, it is envisioned that any suitable display screen and/or format thereof may be utilized in accordance with the present disclosure.

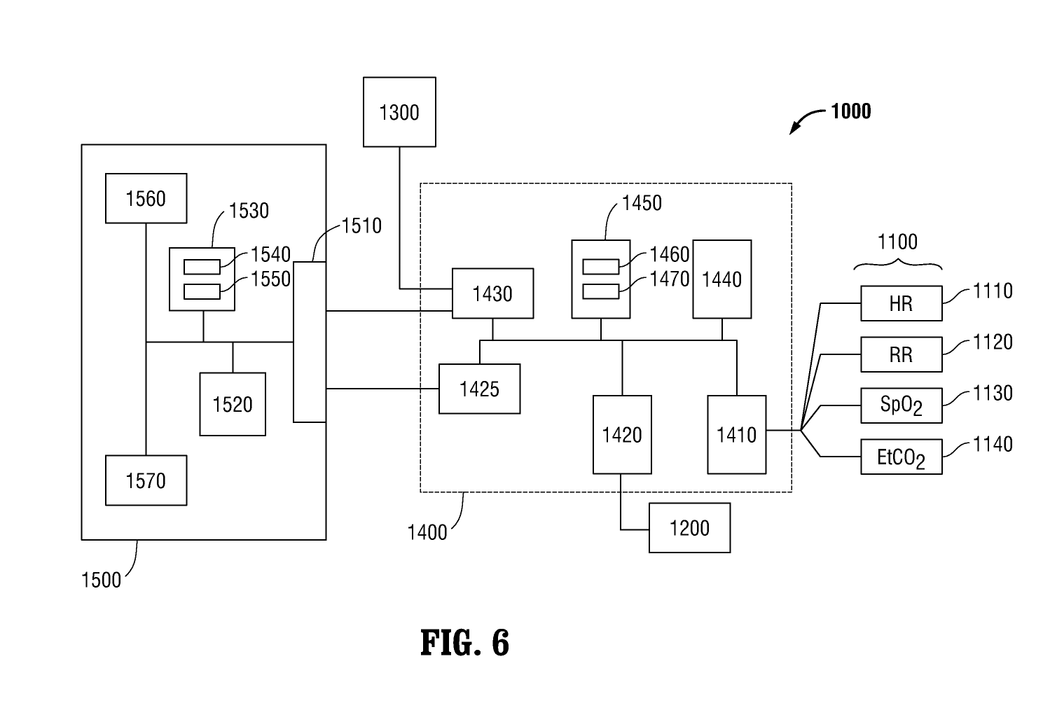

With reference to FIG. 6, another system provided in accordance with the present disclosure and configured for monitoring a patient's cardiopulmonary status and providing feedback based control in accordance therewith is shown generally as system 1000. System 1000 is similar to and may include any of the features of system 10 (FIG. 1), detailed above. Accordingly, only the differences between system 10 (FIG. 1) and system 1000 will be described in detail below for purposes of brevity.

System 1000 includes: a plurality of sensors 1100, e.g., a heart rate monitor 1110, a respiratory rate monitor 1120, a pulse oximeter 1130, and a capnography sensor 1140; one or more input devices 1200, one or more output devices 1300, a processing module 1400, and one or more controllable or programmable apparatus such as, for example, an exercise apparatus 1500. The processing module 1400 of system 1000 includes a sensor input 1410, a device input 1420, an exercise apparatus input 1425, an output 1430, a storage device 1440, and a controller 1450 including a processor 1460 and a memory 1470. Exercise apparatus input 1425 is configured to receive operational data from exercise apparatus 1500 including, for example, a state of the exercise apparatus 1500, e.g., whether the apparatus 1500 is operating or idle, and data regarding the operational settings of the apparatus 1500, if currently operating. As can be appreciated, the operational data may vary depending on the particular exercise apparatus used. With respect to a treadmill, for example, such operational data may include current and past speed settings, incline settings, an exercise program being utilized, etc.

Exercise apparatus 1500 includes: an input/output 1510; a storage 1520; a controller 1530 including a processor 1540 and a memory 1550; an on/off unit 1560, and a motor unit 1570. Alternatively, exercise apparatus 1500 may share its storage and controller with processing module 1400 and, thus, separate storages and controllers need not be provided.

Input/output 1510 is configured to communicate with exercise apparatus input 1425 and output 1430 of processing module 1400. More specifically, input/output 1510 provides operational data to exercise apparatus input 1425 of processing module 1400 and receives control signals from output 1430 of processing module 1400. On/off unit 1560 control the start-up of exercise apparatus 1500, e.g., upon activation of an on-off switch, as well as the shutdown or temporary pausing of exercise apparatus 1500, e.g., as a result of a detected error, as a safety function, or as directed by processing module 1400. Motor unit 1570 controls the motor of the exercise apparatus 1500 in accordance with the settings selected by the user, the particular exercise program being operated, or as directed by processing module 1400. For example, with respect to a treadmill, motor unit 1570 controls the speed at which the belt is driven, the angle of inclination of the belt, etc.

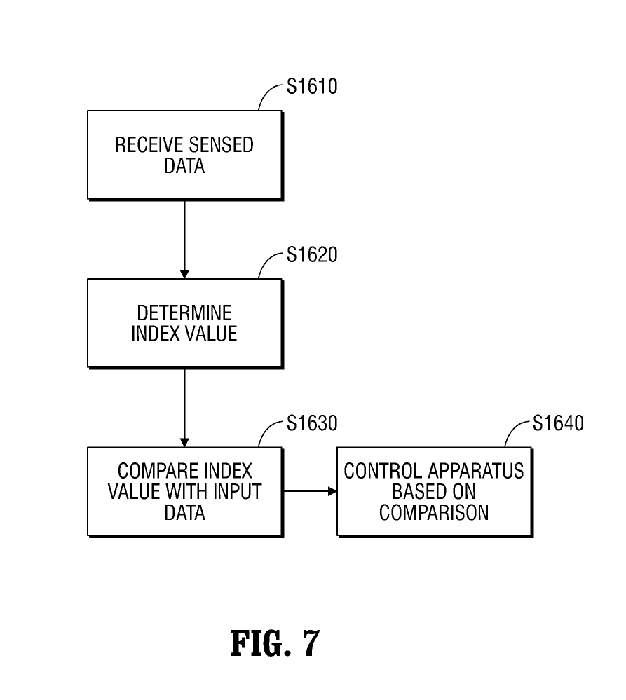

With additional reference to FIG. 7, controller 1450 of processing module 1400 is configured to receive the measured data from sensors 1110, 1120, 1130, 1140 (step S1610); determine an index value based upon the sensed data (step S1620); compare the index value with the input data provided via device input 1420 and/or storage device 1440 (step S1630); and provide control signals to exercise apparatus 1500 for adjusting the operating settings of exercise apparatus 1500, e.g., via motor unit 1570, and/or for stopping the operation of exercise apparatus 1500, e.g., via on/off unit 1560, in accordance with the comparison (step S1640). For example, where the patient's index value is within acceptable limits (as compared to the input data provided or limits calculated via the input data), exercise apparatus 1500 is permitted to continue operation in its present manner; where the patient's index value is bordering on or trending away from acceptable limits, controller 1450 may signal exercise apparatus 1500 to control motor unit 1570 so as to decrease the speed, intensity, and/or other operational settings of exercise apparatus 1500, or to adjust the exercise program currently running; and where the patient's index value is outside of the acceptable limits, controller 1450 may signal exercise apparatus 1500 to control on/off unit 1560 to stop the operation of exercise apparatus 1500 for a pre-determined period or indefinitely. Reactivation of exercise apparatus 1500 and/or increasing the operational settings thereof may be inhibited for a pre-determined period or until the patient's index value returns to within acceptable limits.

As can be appreciated, system 1000 provides automatic feedback and communication between exercise apparatus 1500 and processing component 1400 to limit continued exertion and/or inhibit further exertion in instances where the patient has strayed from his or her optimal exercise zone. System 1000 may provide such automatically control, or may additionally provided suitable output for displaying or audibly producing the patient's index value and/or status indicators, alerts, notifications, etc. regarding the same, as detailed above.

Systems 10, 1000 (FIGS. 1 and 6, respectively) may further be configured to output notifications and/or reports. For example, notifications may be provided to notify medical personnel that the patient's index value dropped below acceptable limits. Reports may be provided to detail the results of a 6-minute walk test, as a periodic update on the patient's condition, for use in determining a suitable exercise program for the patient or similar patients, to fulfill reporting requirements, for inclusion in the patient's EMR file and/or an ADT electronic file, etc. The notifications and/or reports may be provided in any suitable form, e.g., as a visual display, audible alert, data save, print-out, message (text, email, voicemail, etc.) and may be local and/or remote, e.g., transmitted across systems 10, 1000, broadcast, etc.

While several embodiments of the disclosure have been shown in the drawings and described in detail hereinabove, it is not intended that the disclosure be limited thereto, as it is intended that the disclosure be as broad in scope as the art will allow. Therefore, the above description and appended drawings should not be construed as limiting, but merely as exemplifications of particular embodiments. Those skilled in the art will envision other modifications within the scope and spirit of the claims appended hereto.

What is claimed is:

1. A system, comprising:
a plurality of sensors, each sensor configured to measure at least one cardiopulmonary parameter and to provide measured data relating to the at least one cardiopulmonary parameter;
at least one input device configured to provide input data indicating a target cardiopulmonary status;
a monitoring device configured to:
receive the measured data from each of the plurality of sensors,
receive the input data from the at least one input device,
determine a cardiopulmonary status based upon the measured data,
compare the determined cardiopulmonary status with the input data, and
provide a result signal based upon the comparison; and
at least one output device configured to receive the result signal and indicate a relationship between the determined cardiopulmonary status and the target cardiopulmonary status.

2. The system according to claim 1, wherein the at least one output device includes a display configured to visually display the indication of the relationship between the determined cardiopulmonary status and the target cardiopulmonary status.

3. The system according to claim 1, wherein the at least one output device includes a speaker configured to audibly output the indication of the relationship between the determined cardiopulmonary status and the target cardiopulmonary status.

4. The system according to claim 1, wherein the plurality of sensors includes at least one of:
a heart rate monitor;
a respiratory rate monitor;
a capnography sensor; and
a pulse oximeter.

5. The system according to claim 1, wherein the plurality of sensors provide the measured data to the monitoring device wirelessly.

6. The system according to claim 1, wherein the monitoring device is integrated into an exercise apparatus.

7. The system according to claim 6, wherein at least one of the output devices is integrated into the exercise apparatus, and wherein the indication of the relationship between the determined cardiopulmonary status and the target cardiopulmonary status is visually displayed on a display of the exercise apparatus.

8. The system according to claim 1, wherein the determined cardiopulmonary status is an index value.

9. The system according to claim 8, wherein the target cardiopulmonary status is a target index value or a target index value range.

10. The system according to claim 1, wherein the monitoring device provides a control signal for controlling an exercise apparatus based upon the comparison.

11. A non-transitory computer readable recording medium storing computer executable instructions to perform a method, comprising:
determining a cardiopulmonary status of a patient that is using an exercise apparatus;
comparing the determined cardiopulmonary status of the patient with a target cardiopulmonary status; and
controlling the exercise apparatus in accordance with a result of the comparison.

12. The non-transitory computer readable recording medium of claim 11, wherein controlling the exercise apparatus includes at least one of suspending operation of the exercise apparatus and adjusting at least one operational setting of the exercise apparatus.

13. The non-transitory computer readable recording medium of claim 12, wherein the exercise apparatus is a treadmill.

14. The non-transitory computer readable recording medium of claim 11, wherein the cardiopulmonary status is determined based upon data received from at least one sensor configured to measure at least one cardiopulmonary parameter.

15. The non-transitory computer readable recording medium of claim 14, wherein the determined cardiopulmonary status is an index value and wherein the target cardiopulmonary status is a target index value or a target index value range.

16. The non-transitory computer readable recording medium of claim 15, wherein cardiopulmonary status is determined based on data received from one or more of a heart rate monitor, a respiratory rate monitor, a capnography sensor, and a pulse oximeter.

17. A method, comprising:
determining a cardiopulmonary status of a patient that is using an exercise apparatus;
comparing the determined cardiopulmonary status of the patient with a target cardiopulmonary status; and
controlling the exercise apparatus in accordance with a result of the comparison.

18. The method according to claim 17, wherein controlling the exercise apparatus includes at least one of suspending operation of the exercise apparatus and adjusting at least one operational setting of the exercise apparatus.

19. The method according to claim 17, wherein the cardiopulmonary status is determined based upon data received from at least one sensor configured to measure at least one cardiopulmonary parameter.

20. The method according to claim 17, wherein the determined cardiopulmonary status is an index value and wherein the target cardiopulmonary status is a target index value or a target index value range.
